(19) United States
(12) Patent Application Publication
(10) Pub. No.: US 2025/0114115 A1
(43) Pub. Date: Apr. 10, 2025

(54) A sound and vibration generator and a system and method for a sound and vibration analysis

(71) Applicant: SURGIFY MEDICAL OY, Espoo (FI)

(72) Inventor: Seyedshahabaddin HAERI, Helsinki (FI)

(73) Assignee: SURGIFY MEDICAL OY, Espoo (FI)

(21) Appl. No.: 18/730,045

(22) PCT Filed: Mar. 2, 2023
(86) PCT No.: PCT/FI2023/050118

§ 371 Date: Jul. 18, 2024

(30) Foreign Application Priority Data
Mar. 2, 2022 (FI) FI20227030

(51) Int. Cl. A61B 17/32; A61B 17/00; A61B 90/00

(52) U.S. Cl. CPC A61B 17/32; A61B 90/06; A61B 90/08; A61B 2017/00075; A61B 2017/320008; A61B 2090/064; A61B 2090/08021

(57) ABSTRACT

A sound and vibration generator, a method and system for a sound and vibration analysis of processing of a material during a surgery. The sound and vibration generator has one or more vibratory elements arranged to vibrate and at least partially incorporated into a processing means of a surgical device. The system and method is arranged to generate a sound and a vibration by a signal and vibration generator, receive the generated sound and vibration by one or more signal receivers, process the generated sound and vibration to determine at least one of a pre-breakthrough phase, a breakthrough event, a transition moment from a first layer of the material to a second layer of the material or a location of the processing means.

### TECHNICAL FIELD

The present disclosure generally relates to a sound and vibration analysis, more specifically sound and vibration analysis of processing of a material during a surgery.

### BACKGROUND

In bone-cutting surgical procedures the sensitive tissues, usually soft, can be compromised or damaged. A dangerous occasion that is called a breakthrough event is when the cutting tool penetrates the space on the other side of the bone or inside the bone channels and cavities. In these cases, due to limited visual access, the surgeon has no understanding of the situation and might not be able to respond in time to avoid contact with and damage to soft and sensitive tissues.

In the recent past, advances have been made to utilize the processing of sound and vibrations of the bone. The technical challenge with this approach has been that conventional surgical devices such as burrs and saws are originally designed and constantly developed for removing bone with minimal vibration and sound generation or attempts have been made to reduce vibration as a disturbing factor. In addition, the signal produced from the processing of bone is transient and depends on multiple factors.

Cutting bone creates vibrations resulting in the generation of acoustic and vibrational signals which can be used for characterization of the bone type and breakthrough detection. It is known that during the surgery, the sounds and vibrations are not controllable, thus the sound analysis of the hard tissue processing during the surgery is not precise.

Therefore there is a need for controllable, safe and precise techniques to prevent damaging soft, sensitive and unwanted tissues during the surgery.

### SUMMARY

The aim of the present disclosure is to provide a system and a method to prevent damage to soft or sensitive tissues under the bone, or inside the passages and cavities such as blood vessels, nerves, and sinuses.

The aim of the disclosure is achieved by a sound and vibration generator and a method for a sound and vibration analysis of a material during a surgery as defined in the appended independent claims to which reference is made to. Advantageous features are set out in the appended dependent claims.

Processing bone creates vibrations resulting in the generation of acoustic signals which can be used for characterization of the bone type and breakthrough detection. The purpose of the present disclosure is to provide solutions for predicting and/or detecting breakthrough events during bone cutting. In addition, the system and method according to the present disclosure can be utilized for classifying the bone layers, for instance to distinguish between cancellous and cortical bone. Mechanical sound and vibration generators according to the present disclosure are additional signal sources that are utilized for signal processing for determining the location of the surgical device.

Additional advantages of the present disclosure are that the vibrational response of the bone can be processed for assessing the condition of the bone and other characteristics of the bone, such as density, hardness, and thickness.

During processing of the hard tissue, the processing means penetrates through the one area or layer of the bone and enters the adjacent area or layer of the bone or the soft tissue. To prevent unwanted damages to the hard and soft tissues it is necessary to know when to stop drilling.

This requires determining the location of the processing means in the hard tissue. The present disclosure thus provides a surprisingly efficient solution for generating sound and vibration signals in a controllable manner which are used for preventing damaging soft, sensitive or unwanted tissues during the surgery.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the embodiments of the disclosure are shown in the drawings, with references to the following diagrams wherein:

FIG. 1 illustrates a sound analysis system structure;

FIG. 2 illustrates an example a sound and vibration generator integrated to a burr device;

FIG. 3 shows a head section of a burr device;

FIG. 4 illustrates different cutting scenarios according to an embodiment of the present disclosure;

FIG. 5 illustrates an example of a generated sound and vibration analysis, FFT spectrum of the device according to an embodiment of the present disclosure;

FIG. 6 shows a system for analysing the sound and vibration of processing of a material during surgery;

FIG. 7 shows a table summarizing the signal correlations at different locations of the processing means;

FIG. 8 shows an example of a frequency spectrum analysis of the surgical device comprising a sound and vibration generator;

FIG. 9 shown another example of a frequency spectrum analysis of a conventional cutting burr;

FIG. 10 shows a side view of the surgical device illustrating the sound and vibration generator;

FIG. 11 illustrates a cross-sectional view of the surgical device of FIG. 10 along A-A;

FIG. 12 illustrates a cross-sectional view of the surgical device of FIG. 10 along A-A;

FIG. 13 shows a side view of the surgical device illustrating the sound and vibration generator;

FIG. 14 shows a bottom view of the surgical device illustrating the sound and vibration generator;

FIG. 15 illustrates a cross-sectional view of the surgical device of FIG. 14 along A-A;

FIG. 16 shows a side view of the surgical device illustrating the sound and vibration generator with fixed vibratory elements;

FIG. 17 shows a side view of the surgical device illustrating the sound and vibration generator with fixed vibratory elements.

### DETAILED DESCRIPTION OF EMBODIMENTS

Throughout the present disclosure, the following terms are used in the meaning as follows.

The processing may be cutting, drilling, milling, polishing, sawing, or grinding.

The surgical device may be an oscillating saw, rotary saw, a reciprocating saw, a sagittal saw, a drill bit, a reamer, a mill or a burr. The surgical device may be a burr with processing means comprising a spherical body, egg-like body, pear-like body, conical-shaped body, cylindrical, eggplant-type body, or a combination of the mentioned shapes.

The material processing means may be an oscillating blade, a reciprocating blade, a vibrating blade, a rotating drill bit, a rotating cylindrical burr, a rotating mill, or a rotating burr.

The material may be hard tissue, bone tissue, cartilage tissue, calcified tissue, dental tissue, tendon tissue, ligament tissue, or foreign objects in the body.

The surgery may be orthopedic, neuro, spine, Otolaryngology-Head and Neck, ear, nose and throat (ENT), tooth surgery, etc.

In an embodiment of the present disclosure, the cutting sound can be analyzed by a sound analysis system with the following elements: Microphone: to capture the cutting sounds and convert it to electrical signal; Data acquisitions system: receive the microphone output and convert it to digital/analog signals that can be analyzed; Processor: the combination of hardware and required software for analyzing the signal to e.g. detect the sound frequencies and amplitudes. In addition, this system is responsible for identifying/predicting the breakthrough based on known patterns (e.g. sudden drop in sound amplitude or appearance/disappearance of certain sound frequencies in the FFT spectrum of the signal); User interface: for communicating with the user. A display, indicators, buzzer, vibrator, etc. can be used for this purpose.
Different sources of vibrations/sounds during the bone-cutting process that can be used for analysis are as follows: Friction between rotating cutting edges or grinding surface and the bone;
Chip formation (bone breaking into small pieces); “Prevention means” movements of EP3525691B1 patent; Extra sound-generating features installed at the processing means of the surgical device (e.g., at the tip of the cutter).

It should be noted that the mentioned sound sources can be utilized individually or as a combination for the said purpose. Hence, the scope of this invention shall not be limited to the prevention means movement sounds of the said patent.

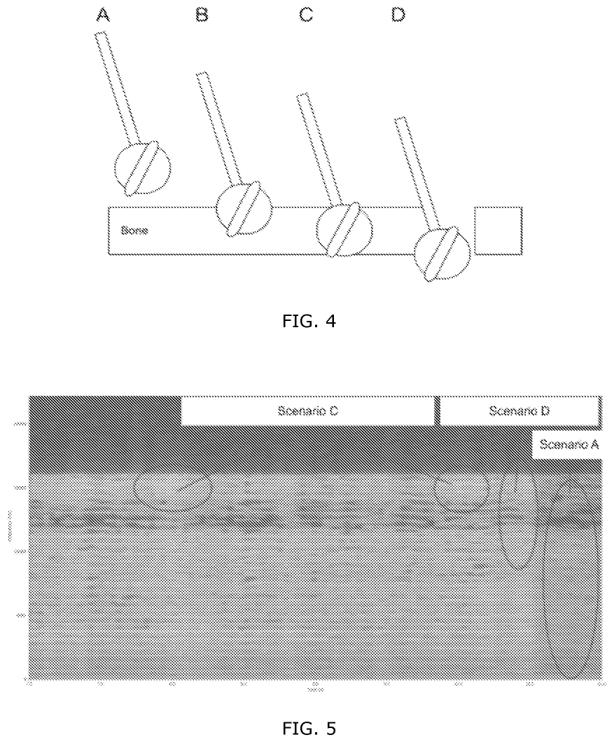

Sound analysis of prevention means. The prevention means sound of different bone-cutting tools can be analyzed for the above-mentioned purposes. In addition, extra sound-generating elements can be added to make a specific noise e.g. when the ring (112) spins or moves radially for more than a specific displacement (e.g. while cutting). In one embodiment of the said patent, for the device (100) the moving ring (112) hits the inner step (202) and generates a distinguishable sound that depends on the bone material, thickness, drill rpm, and forces applied to the drill by the surgeon. Sound signal characteristics such as frequency, amplitude, and their changes carry information about the status of the bone cutting process. The following table and FIG. 4 explain how the noise correlates with the burr head position in relation to the bone.

TABLE 1
Expected sound at different cutting scenarios

| Scenario | Description | Expected sound |
|---|---|---|
| A | There is no contact between | No sound related to cutting |
| | the device and the bone. | process or prevention means |
| | | movement |
| B | The head has penetrated to | Cutting sound and |
| | the bone | prevention means |
| | | sound easily detectable |
| C | The head has approached the | A sudden change in the |
| | bottom of the bone or has | cutting and/or |
| | slightly broken the bottom | prevention means sound |
| | layer | |
| D | The head has penetrated to | Low or no detectable sound |
| | the space beneath the bone | related to cutting process or |
| | layer | prevention means movement |

The sound of different stages of the cutting process with the device (100) is analyzed and its FFT spectrum is shown FIG. 5. The rotating speed of the cutting device is 617 rounds/sec. As a result, corresponding frequencies with the ring movement are multiple of the 617 Hz frequency, i.e. 617, 1234, 1851, 2468, etc.

### Other Sources

As mentioned, the cutting sound of other cutting devices, including the said patent devices and other conventional devices, can be analyzed similarly to the presented example of the previous section with the presented invention. The sound analysis can be combined with other measurements such as cutting force, vibrations, motor torque, or rpm for enhanced reliability.

Thus, a system for characterization of the material type and breakthrough detection during the processing of the material, when the material is selected from a bone, a cartilage, a calcified tissue, a tooth and a foreign object within a patient body, is described according to the present disclosure.

In an aspect, an embodiment of the present disclosure provides a sound and vibration generator for a sound and vibration analysis of the processing of a material during a surgery, wherein the sound and vibration generator comprises one or more vibratory elements arranged to vibrate, and wherein the sound and vibration generator is at least partially incorporated into a processing means of a surgical device.

In an embodiment, the sound and vibration generator is at least partially incorporated into the processing means formed of a grinding surface. In another embodiment, the sound and vibration generator is at least partially incorporated into the processing means formed of one or more cutting edges. Optionally, the sound and vibration generator is incorporated into a cutting edge of the processing means.

The one or more vibratory elements may be arranged to generate sound and vibration by periodically hitting a surface of the material by periodically hitting a surface of the surgical device. The one or more vibratory elements may be fixed on the outer surface of the processing means. The one or more vibratory elements may be movable in relation to the processing means. The one or more vibratory elements are one or more protrusions on the outer surface of the processing means.

The one or more vibratory elements may comprise vibratory elements installed so that the vibratory elements are adapted to reach the material surface prior to the processing means surface. In an embodiment the sound and vibration generator may move in relation to the cutting edge.

The one or more vibratory elements may be arranged to generate the sound and vibration when the processing means is adjacent to the material. The one or more vibratory element may be arranged to generate the sound and vibration when the material is being processed.

In another aspect, an embodiment of the present disclosure provides a system for a sound and vibration analysis of processing of a material during a surgery, wherein the system comprises a surgical device comprising a processing means, a sound and vibration generator at least partially incorporated to the processing means of the surgical device, one or more signal receivers a computing device, a signal measurement means comprising a controller connected to one or more signal receivers and to the computing device and configured to receive the generated signal measured by the one or more signal receivers, convert the signal to a digital form, and transfer the digital signal to the computing device for performing signal processing of the received signal.

An arrangement of one or more signal receivers is selected from in relation to the surgical device or in relation to the material, and wherein the one or more signal receivers is selected from a group comprising an air microphone, a contact microphone, an accelerometer or a force sensor. According to the different embodiments, the arrangement in relation to the material of the one or more signal receivers may be that the one or more signal receivers is attached to the material or installed at the distance from the processing point of the material.

In a third aspect, an embodiment of the present disclosure provides a method for a sound and vibration analysis of the processing of a material during a surgery, the method comprises generating a sound and a vibration by a signal and vibration generator, receiving the generated sound and vibration by one or more signal receivers, processing the generated sound and vibration to determine at least one of a pre-breakthrough phase, a breakthrough event, a transition moment from a first layer of the material to a second layer of the material or a location of the processing means. The at least one of a pre-breakthrough phase, a breakthrough event, a transition moment from a first layer of the material to a second layer of the material or a location of the processing means is determined by determining change in the amplitude of the sound or vibration. The change of the amplitude may be a sudden rise of the amplitude, sudden drop of the amplitude or an amplitude shift. The method may further comprise using an operational speed of a surgical device by the computing device in processing the signal of the generated sound and vibration.

In a fourth aspect, an embodiment of the present disclosure provides a computer program for a sound and vibration analysis of processing of a material during a surgery comprising instructions which when the computer program is executed by a computing device, cause the processor to perform the method when the sound and vibration is generated by a signal and vibration generation means according to the present disclosure.

The use of sound and vibrations generated during the processing of bone with cutting instruments is important for notifying the surgeon or a robot system about the location of the cutting tool in relation to the bone boundaries to prevent damage to soft or sensitive tissues under the bone, or inside the passages and cavities such as blood vessels, nerves, and sinuses. In addition to preserving the sensitive tissue during bone processing, the signal analysis is utilized for improving and optimizing the cutting process. For instance, the bone can be removed with the elevated speed in safe areas, and the speed can be reduced when the bone thickness reduces. These benefits can be utilized in manual and robotic bone-processing procedures in different material processing scenarios such as not cutting (cutting air), far from boundaries, close to boundaries (thin layer formed), the opening made in the bone (breakthrough).

The sounds and vibrations generated during material processing with cutting tools comprising signal and vibration generator according to the embodiments of the present disclosure may be as follows. The cutting sound and vibration of the material (e.g., the bone or other hard tissue) is emitted from the material due to the friction and bone pieces breakage sound and vibration which is the result of interaction between the bone and sharp cutting edges. In such embodiments the signal and vibration generator according to the embodiments of the present disclosure is arranged to act as a signal amplifier. In other embodiments the sound and vibration is generated by the signal and vibration generator according to the present disclosure, e.g. the vibration and sound response of the bone is excited by impact excitation of the signal and vibration generator. The signal and vibration generator is configured to excite the material by impacting it by e.g., every revolution. The sound and vibration emitted in response to impact from the bone is then analyzed. The excitement is done in a controlled and repeatable manner, unlike cutting sound which is random and unreliable as it's affected by multiple factors.

In some embodiments the sound and vibration is emitted by the mechanism of the signal and vibration generator, e.g., generated by the movement of the internal components of the mechanism of the signal and vibration generator. E.g., the rotating or oscillating subcomponents of the signal and vibration generator hit each other with specific frequencies which depend on the rotational or oscillation speed of the motor. The amplitude of these vibrations also depends on the type of material that the generator interacts with.

Throughout the present disclosure, the “at least partially incorporated to” refers to that the sound and vibration generator may be attached to the processing means of a surgical device or the sound and vibration generator may be outer or internal or partially internal part of the processing means.

The known tools do not generate enough cutting vibration due to a very minimal rate of bone removal and fine cutting. Adding the sound and vibration generator according to the present disclosure to such tools makes it possible to analyze the surgical device's location. Different layers of the bone have different characteristics (e.g. damping and hardness) that can be utilized to understand the location of the surgical device as well as assess the material type that is being processed. Furthermore, the relative location of the surgical device compared to the material provides an insight into the break-through situation where the processing means of a surgical device penetrates to the underlying tissues. The addition of the sound and vibration generator provides clear indications in a timely fashion before the breakthrough occurs. This is made possible by exciting vibrations in a controlled manner in the bone.

When the bone becomes thin the clear signal indications are used to predict the occurrence of the break-through.

Moreover, the produced signals during processing and the transient situation between different materials and tissue layers are utilized as location feedback from the processing point in robotic applications.

The processing of the material and interaction of the sound and vibration generator and the material produce vibration both in the material and the surgical device. In addition, the material and moving parts' vibrations produce sound waves by vibrating the air molecules.

There are two sources of sound and vibration during processing and the interaction of the surgical device with the material.

Firstly, during material processing with the moving surgical device, the interaction between the processing means and the material produces sound and vibration. When the material pieces get removed gradually in the form of tiny chips broken off from the material. In addition, the friction between the processing means and the material surface produces vibration and sound.

Secondly, the sound and vibration generator of the surgical device hist (collides or impacts) the material periodically in every operating cycle (such as a revolution of rotary tools or a cycle of reciprocating or oscillating tools) for a certain number of times, depending on the number of vibrating elements and operating speed of the surgical device vibration and sound is generated with a certain frequency. In this method, the vibration excitement is controlled and repeatable, unlike cutting sound, which is random and unreliable as it's affected by multiple factors such as cutting speed, bone characteristics, and operator performance.

For instance, when a rotary surgical device with 2 independent vibratory elements rotates with 60,000 rounds per minute or a rotational frequency of 1000 Hz about the central axis of the surgical device and the vibratory elements impact the material surface 2 times per rotation, vibration and sound at a frequency of 2000 Hz is generated.

The sound and vibration generator comprising vibratory elements interacts with the material independent from the processing means. In some embodiments the vibratory elements are configured as surface protrusions on the processing means surface. In other embodiments the vibratory elements are installed such that they can reach the material surface prior to the processing means surface. This enables the interaction between the vibratory elements and the material even when the processing means (e.g., the head of the burr) is adjacent to the material. Hence, the generation of vibration and sound in the material and the surgical device is not limited to when the material is being processed. In this arrangement, the sound and vibration generator generates sound and vibration in the material and surgical device during the processing and when the processing means is adjacent to the material before and after the processing. The benefit is that the signal analysis system can assess the material condition even without processing the material. In addition, the initiation of this signal can be used as a warning to the surgeon about the approaching of the surgical device to the material in cases with poor visibility or minimally invasive procedures.

Additionally, in robotic systems, this can be used as a notification to the robotic system for calibrating the position of the surgical device compared to the material.

The vibratory elements vibrate the material constantly. When the material becomes very thin (around 0.3 mm) it starts to generate clear signal indications for the processing means. A sudden change in the vibration and sound patterns is detectable in this situation as well as from one layer of the bone to another layer with different characteristics. This effect can be used for determining the location of the processing means near the boundaries to predict the break-through occurrence before and during its occurrence. For instance, the vibration and frequency pattern significantly changes in the transition of the surgical device from cortical to cancellous bone or from tooth enamel to dentin.

The sound and vibration generator comprising vibratory elements vibrates the surgical device simultaneously with the material. The vibratory elements may be fixed or movable. Movable elements are configured to hit a hard surface of the surgical device to generate additional sound and vibration to further facilitate signal analysis. The benefit is that the vibration and sound is amplified, which can facilitate the analysis of the signal and significantly improve the reliability of the signal analysis system. A resilient part such as spring, elastomer, etc may be arranged under the vibratory element to facilitate the movements of the vibratory elements. The vibratory element may be pin shape, ball shape, wire shape, flap shape, ring shape with or without internal protrusion, singular or multiple surface patterns such as dot or spiral and linear patterns or a combination of different shapes. The vibratory element and the contact surface are selected from hard metals to produce optimal sound and vibration.

The sound and vibration generator signal can be processed together or independent from the material processing signal as well the correlation between these two signals provides additional information about the location of the surgical device and the material condition.

As discussed above the sound and vibration generator thus provide an additional surprising effect by amplifying the signals of sound and vibration generated during processing the material and therefore to determine more precisely when to stop processing the material to avoid damage to underlying tissues. Moreover, some variations of surgical devices such as diamond burrs and oscillating saws generate very minimal vibrations that is a challenge.

Signal analysis for predicting the location of the cutting tools in the bone. The amplitude of the signal at frequencies related to impact excitement, the natural frequency of the bone layer, and their harmonics increase significantly when the bone layer becomes thin and reaches its maximum amount before the cracks grow in the thin layer.

The signal analysis system is configured to detect the cracking sounds of the thin layer, which is an additional indication of the breakthrough happening.

The signal indications related to the thin bone layer and breakthrough as well as cutting sounds, start to fade with the progression of the surgical device through the thin layer (breakthrough).

Besides breakthrough detection, it is possible to utilize sound and vibration signals to recognize the type and structure of the materials that are being processed. In addition, the transition from one type of material to another or from one layer of material to another layer can be seen in the signal.

A processing algorithm can utilize the correlation between frequencies and amplitudes at different layers of the bone, to predict the location of the surgical device in the bone and the mechanical characteristics of the material interacting with the cutting tool. Sudden change in the amplitude of the signal frequencies or switching from one frequency band to another enable to determine the location and physical characteristics.

Different types of bone, such as cancellous and cortical bone and bone marrow or enamel and denting in the tooth, have different densities and hardness characteristics that affect their vibrational behaviors. Cortical bone (the dense outer surface of bone that forms a protective layer around the internal cavity) produces significantly higher amplitude vibration and sound compared to the cancellous bone (characterized by its spongy, porous, honeycomb-like structure and is typically found at the ends of a long bone) or bone marrow (a spongy substance found in the center of the bones). The vibrational behaviors of the material and the transitions from one layer to another can be detected in the bone and surgical device vibration and sound.

### DETAILED DESCRIPTION OF THE DRAWINGS

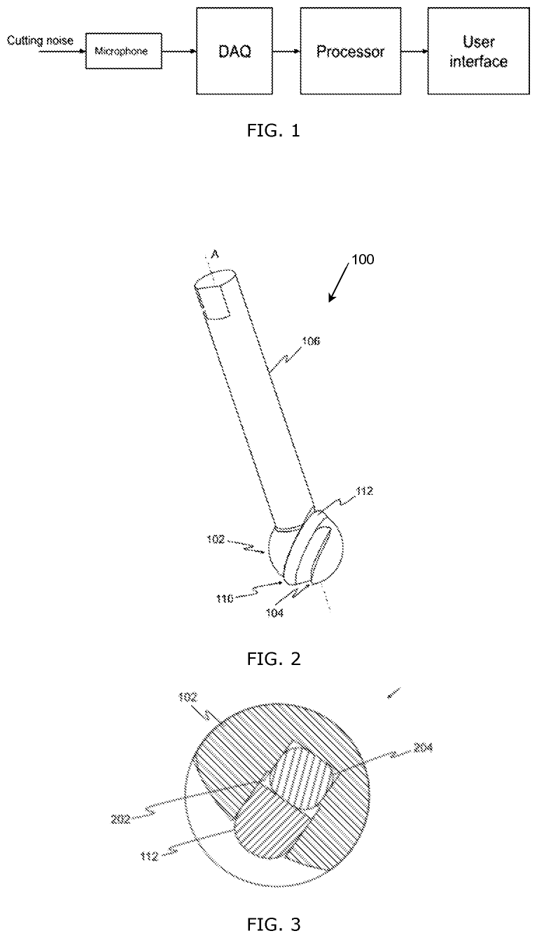

Referring to FIG. 1, there is shown an embodiment of a sound analysis system structure. The sound analysis structure comprises capturing the cutting sounds by a microphone, receiving the microphone output by a data acquisitions system (DAQ) and converting it to digital/analog signals that can be analyzed, receiving the digital/analog signals for analyzing the digital/analog signals, providing the results of the analysis to a user interface.

Referring to FIG. 2, there is shown a surgical device 100, in this embodiment a burr device. The surgical device comprises a processing means 102 having at least one cutting edge 104. The processing means has a spherical shape. The processing means is attached to an attachment means 106. Furthermore, a sound and vibration generator 110 is incorporated at least partially into the processing means 102. Moreover, the sound and vibration generator 110 comprises a vibratory element 112, in this embodiment a ring. Furthermore, the axis A illustrates the axis of rotational motion of the surgical device.

Referring to FIG. 3, there is shown a Head section of a burr device indicating the vibratory element 112 as a moving ring and an inner step 202. The vibratory element 112 vibrates during interaction with a material and periodically hits the step 202. The impact between the vibratory element 112 and step 202 generates sound and vibrations in the surgical tool. Furthermore, the impact between the vibratory element 112 and the material generates sound and vibrations in the material. A resilient element 204 arranged between the vibratory element 112 and processing means 102 facilitate the vibratory movement of the vibratory element 112.

Referring to FIG. 4, illustrates an example of different cutting scenarios, i.e., phases, according to an embodiment. A surgical device, in this embodiment a surgical burr, processing means location in relation to a bone boundaries is shown. In the phase A, there is no contact between the surgical device and the bone and no sound related to cutting process or prevention means movement is generated by a sound and vibration generator. In the phase B the head of the surgical device has penetrated to the bone, cutting sound and prevention means sound easily detectable. In the phase C, the head has approached the bottom of the bone or has slightly broken the bottom layer, a sudden change in the cutting and/or prevention means sound. In phase D, the head has penetrated to the space beneath the bone layer, low or no detectable sound related to cutting process or prevention means movement.

Referring to FIG. 5, there is shown a FFT spectrum of the device 100. The sound amplitude at frequencies that correspond to the ring movement (vibratory element 112) radically changes at different stages of the cutting process. The amplitude is shown by the color density and darker color represents higher amplitude.

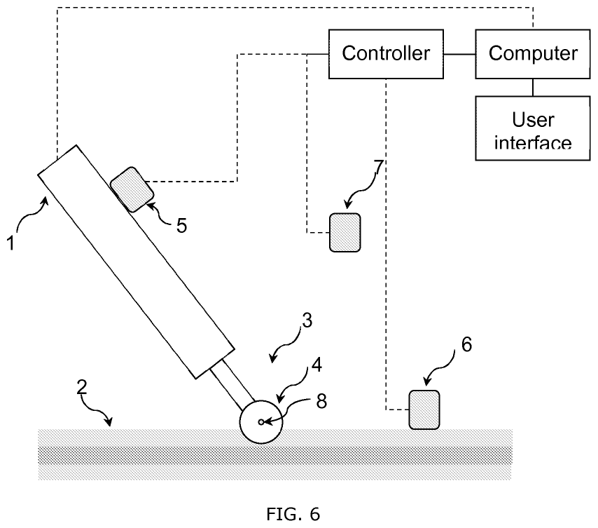

Referring to FIG. 6, there is shown a system for analysing the sound and vibration of processing of a material 2 during surgery. A measurement means receiving the generated signal measured by signal receivers, converting the signal to a digital form, and transferring it to a computer system 11. Computer system 11 processes the received information and conducts mathematical signal processing via dedicated software. The surgical device's operational speed may be used by the processing software for a more accurate assessment of the situation. The outcome of the processing is communicated with the operator (surgeon) or the robot controller system.

The signal measurement means comprises a controller device 10 connected to the power tool 1 and one or more signal receivers 5, 6, and 7 selected from an air microphone, contact microphone, accelerometer, and force sensors arranged in different locations in relation to the surgical device 3 and material 2.

First signal receiver 5 attached to the power tool 1, provides a detectable signal related to sound and vibration generator transferred through the surgical device 3. Second signal receiver 6 attached to the material measures the vibrations generated by the processing means and sound and vibration generator transferred through the material. Third signal receiver 7 installed at a distance, for example, 10 to 80 cm from the processing point, measures the sound emitted by the material and sound and vibration generator. Third signal receiver 7 may be installed on the power tool 1, material surface, surgeon's or patient's body, or other locations. A computer program for a sound and vibration analysis of generated sound and vibrations during a surgery comprising instructions which when the computer program is executed by a computing device, enables the processor to perform the method.

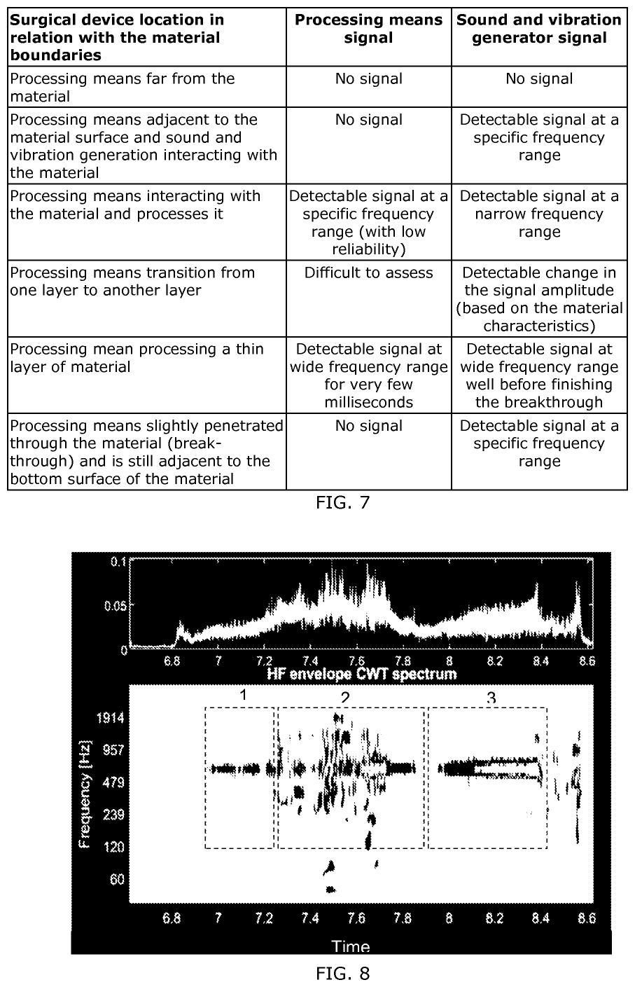

Referring to FIG. 7, another example of phases of sound and vibration generation according to another embodiment of the present disclosure.

Referring to FIG. 8, there is shown a frequency spectrum analysis of the surgical device comprising a sound and vibration generator rotating at 615 Hz. A significant rise in the amplitude of a narrow frequency range around the rotating frequency of the tool indicates the location of the processing means near the boundary of the bone (thin layer bone is formation) shown in dashed window 1. The sound and vibration generator excites the bone layer for a long period of bone for around 0.6 sec before the processing means breaks through the bone (shown in dashed window 2). The sound and vibration generator continues generating signals in a narrow range of frequencies after the breakthrough for around 0.5 sec, as shown in dashed window 3. The combination of the clear sound and vibration generated by the sound and vibration generator over 1.3 sec time provides enough reliability and reaction time for the operator or robotic system to react to the approaching event of bone breakthrough.

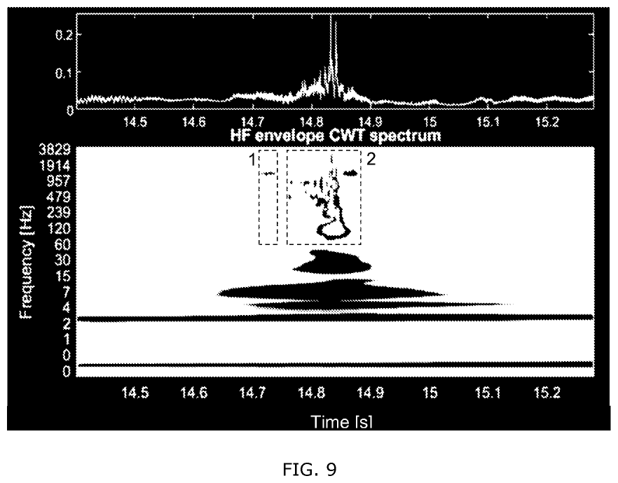

Referring to FIG. 9, there is shown a frequency spectrum analysis of a conventional cutting burr with cutting edges rotating at 1250 Hz. A significant rise in the amplitude of a narrow frequencies range around the rotating frequency of the tool (showed in dashed window 1) followed by a sudden rise in the amplitude of the signal over a wide range of frequencies for around 0.1 sec (shown in dashed window 2) indicates the breakage of the think layer (break-thorough). The identification of the processing means located near the boundary of the bone has become extremely difficult due to the lack of time to respond from the first detectable signal (less than 0.2 sec). In addition, the detected indications are less reliable as the processing system relies on the processing sound and vibrations that are sensitive to bone characteristics.

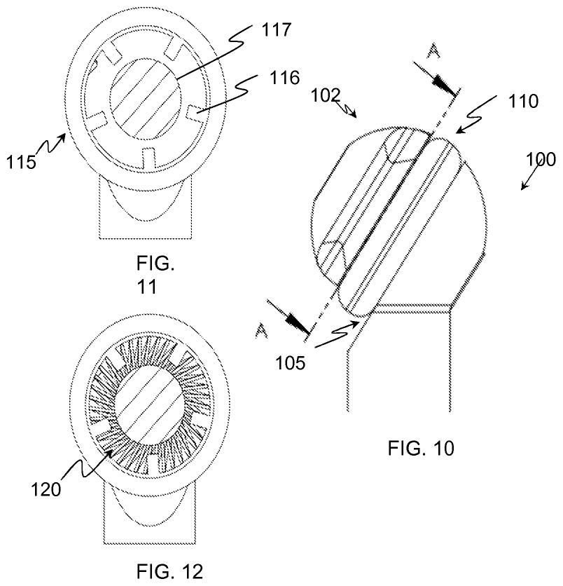

Referring to FIG. 10, there is shown a side view of the surgical device 100 illustrating the sound and vibration generator 110 arranged in an indentation 105. The indentation 105 is arranged along the surface of the processing means 102 to receive the sound and vibration generator 110.

Referring to FIG. 11, illustrated is a cross-sectional view of the surgical device 100 of FIG. 10 along A-A. As shown, the surgical device 100 includes an indentation for incorporating the sound and vibration generator 110. The sound and vibration generator comprises a vibratory element 115. Moreover, vibratory element 115 includes at least one internal protrusion 116 that is arranged to generate vibration and sound via hitting the inner surface 117 of the indentation.

Referring to FIG. 12, illustrated is a cross-sectional view of the surgical device 100 of FIG. 10 along A-A. A resilient part in the form to spring 120 is arranged inside the indentation 105 under vibratory element 115 to facilitate the movement of the vibratory element 115.

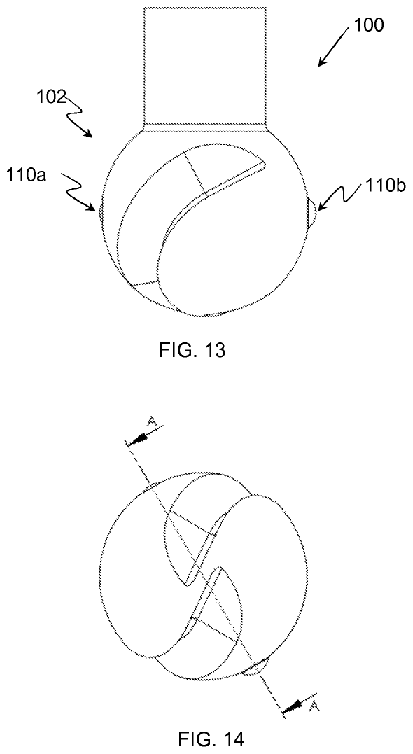

Referring to FIG. 13, there is shown a side view of the surgical device 100 illustrating the sound and vibration generator 110a and 110b arranged in the processing means 102.

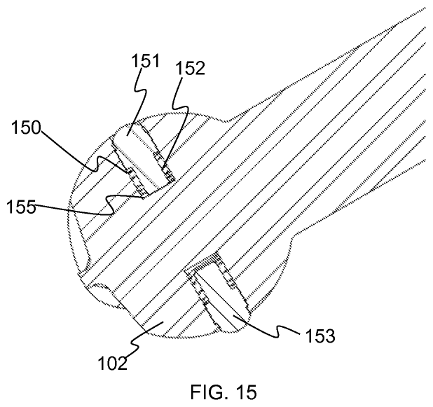

Referring to FIG. 14, there is shown a bottom view of the surgical device 100 of FIG. 13.

Referring to FIG. 15, illustrated is a cross-sectional view of the surgical device 100 of FIG. 140 along A-A. As shown, the surgical device 100 includes at least one indentation 150 in form of a hole into processing means 102 for incorporating at least one sound and vibration generator 110a. The sound and vibration generator 110a comprises a vibratory element 151. A resilient material in form of spring 152 is arranged inside the indentation 150 to facilitate the movement of the vibratory element 151. The vibratory element 151 is illustrated in its inwards position hitting the inner surface 155 of the indentation 150. Moreover, the vibratory element 153 is illustrated in its outwards position before hitting a material.

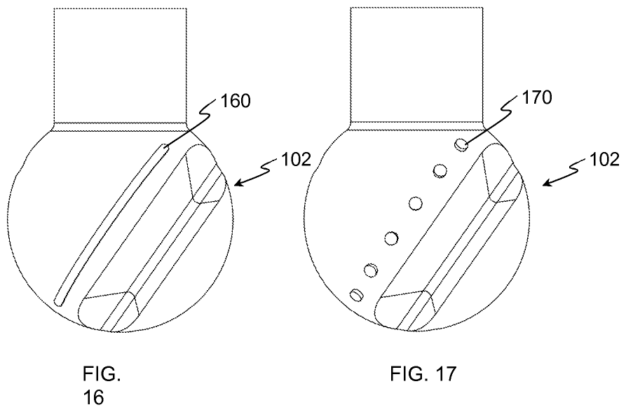

Referring to FIG. 16 and FIG. 17, there is shown additional alternative embodiments of the present disclosure. The surgical device 100 illustrating the sound and vibration generator 160 and 170 arranged as surface protrusions on the outer surface of the processing means 102.

1. A sound and vibration generator for a sound and vibration analysis of processing of a material during a surgery, wherein the sound and vibration generator and comprises
one or more mechanical vibratory elements arranged to vibrate,
the sound and vibration generator is at least partially incorporated into a processing means of a surgical device, and wherein
the sound and vibration generator is configured to amplify the signal of sound and vibration generated during processing of the material by the processing means.

2. The sound and vibration generator according to claim 1, wherein the sound and vibration generator is at least partially incorporated into the processing means formed of a grinding surface.

3. The sound and vibration generator according to claim 1, wherein the sound and vibration generator is at least partially incorporated into the processing means formed of one or more cutting edges.

4. The sound and vibration generator according to claim 1, wherein the sound and vibration generator is incorporated into a cutting edge of the processing means.

5. The sound and vibration generator according to claim 1, wherein the one or more mechanical vibratory elements is arranged to generate sound and vibration by periodically hitting a surface of the material or by periodically hitting a surface of the surgical device.

6. The sound and vibration generator according to claim 1, wherein the one or more mechanical vibratory elements is fixed on the outer surface of the processing means.

7. The sound and vibration generator according to claim 1, wherein the one or more mechanical vibratory elements is movable in relation to the processing means.

8. The sound and vibration generator according to claim 1, wherein the one or more mechanical vibratory elements are one or more protrusions on the outer surface of the processing means.

9. The sound and vibration generator according to claim 5, wherein the one or more mechanical vibratory elements are installed so that the mechanical vibratory elements are adapted to reach the material surface prior to the processing means surface.

10. The sound and vibration generator according to claim 9, wherein the sound and vibration generator may move in relation to the cutting edge.

11. The sound and vibration generator according to claim 1, wherein the one or more mechanical vibratory elements is arranged to generate the sound and vibration when the processing means is adjacent to the material.

12. The sound and vibration generator according to claim 1, wherein the one or more vibratory element is arranged to generate the sound and vibration when the material is being processed.

13. A system for a sound and vibration analysis of processing of a material during a surgery, wherein the system comprises
a surgical device comprising a processing means,
a sound and vibration generator according to claim 1 at least partially incorporated to the processing means of the surgical device,
one or more signal receivers,
a computing device,
a signal measurement means comprising a controller connected to one or more signal receivers and to the computing device and configured to receive the generated signal measured by the one or more signal receivers, convert the signal to a digital form, and transfer the digital signal to the computing device for performing signal processing of the received signal.

14. The system according to claim 13, wherein an arrangement of one or more signal receivers is selected from in relation to the surgical device or in relation to the material, and wherein the one or more signal receivers is selected from a group comprising an air microphone, a contact microphone, an accelerometer or a force sensor.

15. The system according to claim 13, wherein the computing device is configured to process the generated sound and vibration to determine at least one of a pre-breakthrough phase, a breakthrough event, a transition moment from a first layer of the material to a second layer of the material or a location of the processing means.

16. The system according to claim 13, wherein the computing device is further configured to use an operational speed of a processing device to aid in processing the signals of the generated sound and vibration.

17. A computer program for a sound and vibration analysis of processing of a material during a surgery comprising instructions which when the computer program is executed by a computing device of a system according to claim 13, cause the computing device to analyze sound and vibration generated by a signal and vibration generator and to analyze an amplified signal of sound and vibration generated during processing of material by a processing means of a surgical device.

18. The computer program product according to claim 17, wherein the vibration generator comprises one or more mechanical vibratory elements arranged to vibrate, the vibration generator being at least partially incorporated into the processing means of the surgical device, and wherein the vibration generator is configured to amplify the signal of sound and vibration generated during processing of the material by the processing means
